(19) United States
(12) Patent Application Publication
(10) Pub. No.: US 2024/0407671 A1
(43) Pub. Date: Dec. 12, 2024

(54) METHOD AND SYSTEM FOR ASSESSMENT OF CLINICAL AND BEHAVIORAL FUNCTION USING PASSIVE BEHAVIOR MONITORING

(71) Applicant: Seth Feuerstein, New Haven, CT (US)

(72) Inventor: Seth Feuerstein, New Haven, CT (US)

(21) Appl. No.: 18/805,002

(22) Filed: Aug. 14, 2024

Related U.S. Application Data
(63) Continuation of application No. 17/097,787, filed on Nov. 13, 2020, now abandoned.
(60) Provisional application No. 62/936,059, filed on Nov. 15, 2019.

(51) Int. Cl. A61B 5/11; A61B 5/00; A61B 5/01; A61B 5/021; A61B 5/024; A61B 5/145; A61B 5/369; G16H 10/60; G16H 50/30

(52) U.S. Cl. CPC A61B 5/1128; A61B 5/0013; A61B 5/01; A61B 5/021; A61B 5/024; A61B 5/1127; A61B 5/14532; A61B 5/369; A61B 5/4815; A61B 5/4848; A61B 5/4872; A61B 5/4884; A61B 5/7405; G16H 10/60; G16H 50/30

(57) ABSTRACT

A system and method for capturing and analyzing a person's behavioral functions (e.g., 3-D bodily movements, facial expressions, vocalizations), and for developing a corresponding personal behavioral signature. The capture may be performed passively, e.g., in contactless manner (e.g., using LIDAR or camera-based imaging, and/or a microphone). Artificial intelligence, machine learning, and/or other techniques to may be used to analyze captured behavioral function data to identify patterns, assess characteristics, etc., and develop the corresponding behavioral signature. A baseline signature may be compared to a subsequent signature to assess a change in behavioral state. Similarly, data and/or signatures for multiple individuals having a common disease (or receiving a common treatment) may be analyzed to develop a characteristic disease signature (or treatment signature). A person's behavioral signature may be compared to pre-defined disease signatures (or treatment signatures) to identify whether the person has the disease (or is responding appropriately to the treatment).

### CROSS-REFERENCE TO RELATED APPLICATION

This application claims the benefit of priority, under 35 U.S.C. § 119 (e), of U.S. Provisional Patent Application No. 62/936,059, filed Nov. 15, 2019, the entire disclosure of which is hereby incorporated herein by reference.

### FIELD OF THE INVENTION

The present invention relates generally to computing a person's behavioral and health status and function and creating and comparing respective signature profiles, and more specifically, unobtrusive or remote assessment of behavioral and health function using electronic devices to monitor the person's behavior passively.

### DISCUSSION OF RELATED ART

Behavioral function tests have attempted to measure a person's behavioral status and functional situations across a broad range of clinical domains such as mood, anxiety, psychosis, suicidality, obsessions, compulsions, addictions, as well as medication response for each of these. Behavioral and related health and clinical assessments are administered by trained clinicians, requiring face-to-face human interactions and limiting, as a practical matter, how often these clinical assessments can be performed. Even in the most intensive settings, such as an inpatient unit, evaluations such as these occur infrequently, e.g., once per day. They are also subjective. The evaluations can also vary greatly due to a change in who performs the evaluation and also changes in the person's health or behavior due to illness changes or medication and treatment response. Where new attempts have been made to perform such tests, these approaches have required active participation of the person, including wearing and/or operation of monitoring hardware, and/or use of computing hardware (such as a smartphone) for gathering activity data.

Behavioral health is a key part of health that impacts diseases ranging from depression to diabetes to fall risk. It is also important for optimal performance at work and for lessening burdens on society.

Despite the importance of behavioral health, development of reliable systems that remove subjectivity and are scalable and cost-effective has been elusive, especially ones that can do so for repeated and regular assessment in various environments and/or situations, including in a person's home or in a hospital. Additionally, ensuring inter-rater reliability/consistency across evaluations has proven challenging. Often, different clinicians draw different conclusions and/or develop different and/or inconsistent evaluations. Similarly, the emergence of discrete and narrow tool sets tests available through emerging vendors such as Apple, e.g., via the Apple Watch sold by Apple Inc. of Cupertino, California, and Mindstrong of Palo Alto, California, via its smartphone app for monitoring typing behavior on a smartphone, are extremely limited in the types of data they gather, the links they have to clinical outcomes and the severity of the clinical syndromes and situations they can measure.

Other variables also affecting behavior include medications and response, external factors such as stresses, trauma, diet, medical diseases and sleep.

Some parties have looked to mobile devices, such as smartphones, and the various data they can collect. ‘Wearables’ such as watches, clothing, and glasses are also capable of delivering much of the functionality found in a smartphone.

Ginger.io is a company that has looked to this useful but narrow type of data and has reported an application on a smartphone computing device that monitors data about the number and frequency of calls, text messages, and emails sent, and uses the device's global positioning system (GPS) and accelerometer to infer activity level. When a patient deviates from their routine behavior of calling and texting patterns, Ginger.io alerts the individual's caregiver to intervene and assess the situation for noncompliance with medications, inappropriate titration of medications, and other factors that may precipitate a flare-up of the patient's disease. This approach is limited to a narrow type of behavior based on interaction activity via the smartphone hardware, and is based on what is known about that user. This approach does not measure the data in severe states of illness, or define an initial behavioral state (before patterns for that person are known), or combine such data with other data sources or systems having data indicative of patterns. While the present invention can utilize such types of data, it does not rely upon or require it.

Guiding bodies are beginning to see the low value of current systems of diagnosis, tracking and treatment monitoring. For instance, in Belgium it has been recognized that the DSM is of little value in the diagnosis and classification of mental health problems, and in the USA there has been recognition that current diagnostic methods are flawed.

What is needed is a more comprehensive method and system for defining human patterns of healthy and disease behavior in populations, based on monitoring of the behavior of individuals alone, and for defining human patterns and health and disease behavior on an individual basis, and assessing disease state and function in a manner that is highly-sensitive, specific, and unobtrusive to an individual. Also needed are a method and system for providing full three-dimensional views of behavior in a moment in time and over time, and for comparing changes therebetween.

Additionally, a method and system are needed that can predict readiness for a next type of care (for instance, discharge from a hospital or whether a patient should get a certain medication or any medication), as well as how the patient is responding to this care. Additionally, a method and system are needed that can infer risk for future events, such as falling (such as from a recently prescribed medication side-effect), suicide and others. Additionally, a method and system are needed that can provide for adaptive treatment interventions, by providing software-based feedback that monitors behavioral signatures and informs a change in treatment based on the observed behavioral signatures.

### SUMMARY

The present invention provides a system and method configured to capture and analyze/measure a person's behavioral function(s) across multiple physical/spatial dimensions (e.g., 2-D or 3-D) and/or other dimensions (e.g., 4-D, i.e., 3-D over time), and to define/develop one or more personal multidimensional behavioral signatures. In certain embodiments, the present invention captures voice and/or facial movement/expression aspects of the person's behavioral function(s).

In preferred embodiments, the system and method provide for capture and analysis/measurement of the person's behavioral function(s) in a passive manner, without explicit input required by the person/subject, such as participation in clinical interviews, responding to questionnaires, submission to “lab”/bloodwork tests, etc. In a certain preferred embodiment, the system and method provide for capture and analysis/measurement of the person's behavioral function(s) in a contactless manner that does not require the person to wear a fitness tracker or other wearable electronic/computing device or other type of active monitoring device on the person's body.

In one such contactless capture embodiment, an image capture/imaging system is used to capture the person's behavioral functions, such as bodily movements in three-dimensional space, e.g., using a three-dimensional imaging system, such as LIDAR. Alternatively, other imaging systems, such as a video camera capturing two-dimensional images, may be used to perform essentially contactless monitoring of bodily movements in two-dimensions, and computerized image processing may be used to interpret the two-dimension video to assess three-dimensional movements. This may also be leveraged with software and multiple two-dimensional images to interpret the images to provide three-dimensional data.

In certain contactless capture embodiments, an audio capture system (e.g., one including a microphone) is used to capture the person's behavioral functions, such as speech or vocalizations. Data captured in accordance with the present invention may be further combined with other data gathered passively and unobtrusively, e.g., from whatever the user's other activities may be (e.g., interacting with a computer or smartphone to send an email) to develop a behavioral signature in accordance with the present invention. Further, data captured in accordance with the present invention may be further combined with other data gathered using body-contacting devices (such as “fitness trackers” and other wearable devices) and/or data gathered as the results of active, purpose-specific activities (e.g., participation in interviews, questionnaires, etc.) and/or active, non-purpose-specific activities (e.g., activities of daily living, including monitoring the user's other activities, such as interacting with a computer or smartphone, e.g., to send an e-mail or text, browse the internet/Web, etc.) to develop a behavioral signature in accordance with the present invention.

In accordance with the present invention, the system and method provided uses software, artificial intelligence, machine learning, and/or other techniques to analyze captured bodily movements and/or other behavioral functions to identify patterns, assess characteristics, etc. that define a then-current behavioral signature for a particular person. In this manner, one or more personal behavioral signatures is created for each person, e.g., a signature including a three-dimensional motion signature component of the person's actually observed three-dimensional bodily movements.

The system is then used to monitor the person to gather additional behavioral data over time. The system and method compare subsequently observed behavior to the previously-obtained baseline/signature to identify a change (or delta) in the behavior, e.g., by comparing baseline and subsequent behavioral signatures. These objectively-observed changes are used by the system to define and evaluate disease state. Notably, all of this can be done passively, without disrupting the user's day-to-day activities, or without the user's active use of electronic devices.

Further, the present invention enables the identification/creation and detection of new types of signatures of disease, so that diseases can be identified from objectively-observed bodily movements and/or other behavioral functions.

Further still, the present invention enables the identification/creation and detection of new types of drug/treatment signatures, as reflected in the objectively-observed bodily movements and/or other behavioral functions.

Accordingly, the present invention provides a system and method that enables the creation of personal digital behavioral signatures, as well as characteristic behavioral signature profiles associated with a certain diagnosis, a condition change or a treatment response, and further quantifies a baseline and an impact of changes of mobility, physical activity, cognitive activity, social interaction and diet on cognitive function. The system objectively captures/analyzes/records multivariate items including multiple dimensions of motion, velocity of motion, and interrelatedness of various body movements, as well as voice, language, facial movements and facial patterns, and can link these aspects to other data such as body-attached or unattached motion sensors, and data in medical records such as patient history, medication changes and outcomes. The system also examines the relative timing of these data points and related activities and location of a person's behavior, including aspects of the person's behavior as represented in the person's bodily movements in three-dimensional space, including relatedness of bodily motions, speed/velocity of motions, angles of motion, etc., and their changes in an individual setting and in a setting with a group of individuals. Further, aspects of the person's behavior in three-dimensional space in relation to others and/or their environment may be considered by the system. For example, aspects of the person's behavior involving physical proximity to other people, physical locations of the person, quantity of bodily movement vs quantity of bodily rest and other macro bodily movements may be considered by the system.

### BRIEF DESCRIPTION OF THE FIGURES

For a better understanding of the present invention, reference may be made to the accompanying drawings in which:

FIG. 1 is a system diagram showing an exemplary network computing environment in which the present invention may be employed;

FIG. 2 is a schematic diagram of an exemplary special-purpose Behavior Monitoring System computing device in accordance with an exemplary embodiment of the present invention;

FIG. 3 is a functional description of the behavioral function assessment system in accordance with one embodiment of the present invention; and

FIG. 4 illustrates an exemplary computing environment of the behavioral function assessment system configured in accordance with one embodiment of the present invention.

### DETAILED DESCRIPTION

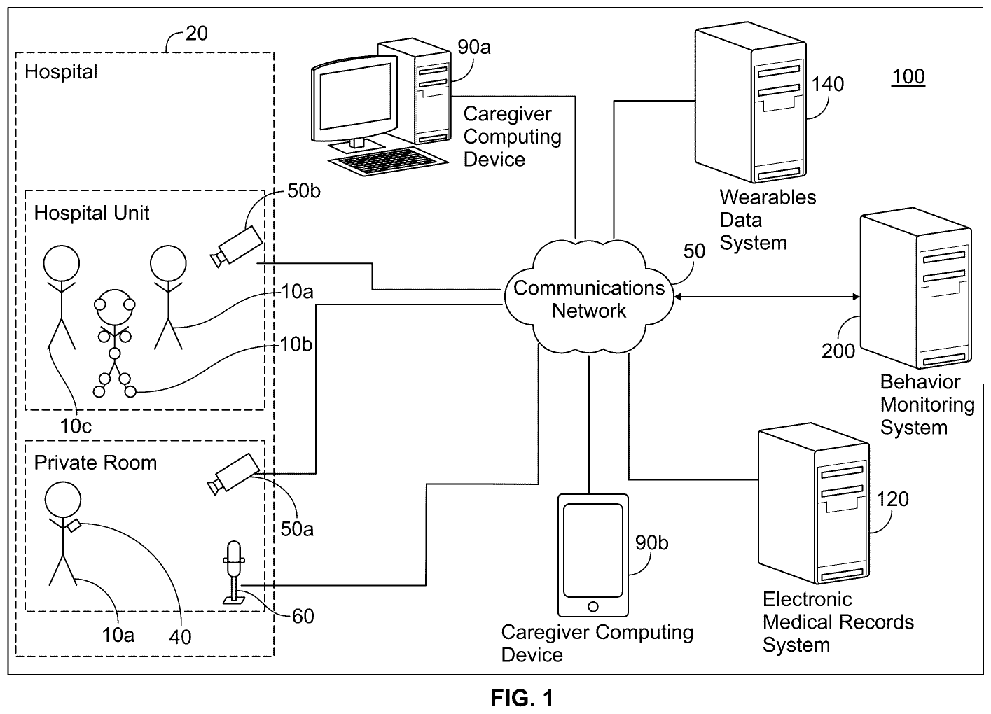

According to illustrative embodiment(s) of the present invention, various views are illustrated in FIGS. 1-4 and like reference numerals are used consistently throughout to refer to like and corresponding parts of the invention for all of the various views and figures of the drawings.

The following detailed description of the invention contains many specifics for the purpose of illustration. Any one of ordinary skill in the art will appreciate that many variations and alterations to the following details are within scope of the invention. Accordingly, the following implementations of the invention are set forth without any loss of generality to, and without imposing limitations upon, the claimed invention.

The present invention provides a system and method configured to capture and analyze/measure a person's behavioral function(s) across multiple physical/spatial dimensions (2-D or 3-D) and/or other dimensions (e.g., 4-D, i.e., 3-D over time), and to develop one or more personal multidimensional (in space or otherwise) behavioral signatures. In preferred embodiments, the system and method provide for capture and measurement of the person's behavioral function(s) in a passive manner, without explicit input required by the person/subject, such as is the case with clinical interviews, questionnaires, lab tests, etc. In a certain preferred embodiment, the system and method provide for capture and analysis/measurement of the person's behavioral function(s) in a contactless manner that does not require the person to wear a fitness tracker or other wearable electronic/computing device or other type of monitoring device on the person's body. As used herein, the references to behavior, behavioral function(s), behavioral pattern(s), etc. are intended to be broad and non-limiting, and specifically to include central nervous system activities and brain-controlled activities (including, for example, emotional activities and physical activities such as gait, balance, and other aspects that may be impacted by side effects of psychiatric or other medications).

### System Environment

An exemplary embodiment of the present invention is discussed below in greater detail for illustrative purposes. FIG. 1 is a system diagram showing an exemplary network computing environment 10 in which the present invention may be employed. As shown in FIG. 1, the exemplary network environment 10 includes conventional computing hardware and software for communicating via a communications network 50, such as the Internet, etc., at the Caregiver Computing Devices 90a (e.g., a personal computer/PC) and 90b (e.g., a tablet computer or smartphone). The system may also include conventional computing hardware and software as part of the Electronic Medical Records System 120, such as an EPIC or Cerner or ALLSCRIPTS system, which may interface with the Caregiver Computing Devices 90a, 90b, as known in the art. Further, the network may include a conventional “fitness tracker” or other “wearable device” 40 that may gather physiological data, such as GPS-type (latitudinal/longitudinal) positional location of the device, heart-rate, blood oximetry, body temperature, electroencephalogram, etc., and communicate that data to a conventional Wearables Data System 140, as known in the art. These systems may be existing or otherwise generally conventional systems including conventional software and web server or other hardware and software for communicating via the communications network 50. Consistent with the present invention, these systems may be configured, in conventional fashion, to communicate/transfer data via the communications network 50 with the Behavior Monitoring System 200 in accordance with and for the purposes of the present invention, as discussed in greater detail below.

In accordance with the present invention, the network computing environment 100 further includes the Behavior Monitoring System (BMS) 200. In this exemplary embodiment, the BMS System 200 is operatively connected to the Caregiver Computing Devices 90a, 90b, and to the other systems shown, for data communication via the communications network 50. For example, the BMS 200 may gather patient-related data from the Caregiver Computing Devices 90a, 90b via the communications network 50. Further, for example, the BMS 200 may gather via the communications network 50 physiological or other data from Wearables Data System 140 that is gathered from a patient's 10a “fitness tracker” or other wearable device 40. Further still, for example, the BMS 200 may gather medical records data from the Electronic Medical Records System 120 via the communications network 50. The gathered data may be used to perform analyses of behavioral function/behavioral signature data, and the results of such analyses may be communicated by the BMS 200 to the Caregiver Computing Devices 90a, 90b, via the communications network 50. Hardware and software for enabling communication of data by such devices via such communications networks are well known in the art and beyond the scope of the present invention, and thus are not discussed in detail herein.

In accordance with the present invention, the network computing environment 100 further includes a contactless behavior capture system. In the exemplary embodiment shown in FIG. 1, the system includes an imaging system for capturing a person's (e.g., patient 10a) behavioral functions, such as bodily movements in three-dimensional space. In the example of FIG. 1, the imaging system is shown as employed in a hospital building 20. More particularly, the example of FIG. 1, shows providing of a three-dimensional motion capture system 50a mounted in a patient's private room. By way of example, the three-dimensional motion capture system may include a LIDAR-type imaging unit 50a for capturing three-dimensional “images” and three-dimensional movements of the patient 10a a within the private room. In this example, the three-dimensional motion capture system 50 a is configured to communicate capture data to the behavior monitoring system 200 via the communications network 50. For example, the three-dimensional motion capture system 50a may be configured to observe the patient's 10a movements within the private room, and to capture data observed from the patient's bodily movements and sub-movements (such as angles of limbs, speed of motion of legs, facial muscle/skin motions and their interrelatedness). Any suitable imaging system capable of three-dimensional capture of bodily movements may be used, and they are preferred as they provide rich data for measurement of bodily movements and sub-movements in three dimensions.

It should be noted that other three-dimensional motion capture systems/Lidar units 50a may be positioned elsewhere within the same building/hospital 20. For example, another Lidar-based motion capture unit may be positioned within a semi-private space where multiple patients may be present, such as within a hospital unit but not within a private room, as will be appreciated from FIG. 1. Further, similar motion capture units may be placed within public areas of the same building/hospital 20 for further capture of the same patient's bodily movements, etc. In this case, the BMS 200 may be provided with software for distinguishing one patient from another, and for aggregating behavioral data captured by multiple different imaging units for a single patient, and then for analyzing the aggregated data for each patient. Any suitable motion capture system may be used for three-dimensional motion capture functions.

By way of alternative example, other imaging systems may be used for two-dimensional motion capture. For example, a video camera 50b for capturing two-dimensional images may be used instead of, or in addition to, three-dimensional motion capture units. Such a video camera 50 may perform essentially contactless monitoring (e.g., by contactless monitoring of a patient wearing markers 70 (or a garment including markers 70) visualizable by the video camera 50. Somewhat similarly, a LIDAR imaging device may perform essentially contactless monitoring (e.g., by contactless monitoring of a patient wearing markers 70 (or a garment including markers 70). Accordingly, in some embodiments, individuals and their movements are identifiable without attaching anything to them. In other embodiments, a patient may wear a marker such as a bracelet or other identifiable item(s) to support capture of their movements and/or to enable each individual to be distinguished from other individuals. Such markers 70 may be used in conjunction with suitable software, e.g., at the BMS 200, for processing two-dimensional images captured by the video camera 50b and interpreting them to produce bodily movements data suitable for the purposes described herein. Such essentially contactless monitoring is considered herein as “contactless”. Two-dimensional imaging systems for capturing two-dimensional bodily movements of the patient may similarly be mounted in a private room and/or in a semiprivate or public space, and the BMS 200 may be provided with software for distinguishing one patient from another, and aggregating behavioral data captured by multiple different imaging units for a single patient and then for analyzing the aggregated data for each patient. Any suitable motion capture system may be used for two-dimensional motion capture.

By way of another alternative example of contactless capture, an audio capture system (e.g., including a microphone 60) may be used to capture the person's behavioral functions, such as speech or vocalizations. Audio capture systems/microphones 60 for capturing audible vocalizations of the patient may similarly be mounted in a private room and/or in a semiprivate or public space, and the BMS 200 may be provided with software for distinguishing one patient's vocalizations from another's, and aggregating behavioral data captured by multiple different audio capture systems for a single patient and then for analyzing the aggregated data for each patient. Any suitable audio capture system may be used. By way of example, the BMS 200 may include voice recognition software, speech analysis software and/or textual analysis software to analyze voice, tone, word choice, speed of speech, quantity of words, length of sentences, use of neologisms, etc. from audio signals captured by the audio capture system to provide additional personal behavioral data that may be aggregated with the personal bodily movement data to develop behavioral signatures, as described herein.

Data captured in accordance with the present invention may be further combined with other data gathered passively and unobtrusively, e.g., from whatever the user's other activities may be (e.g., interacting with a computer or smartphone to send an e-mail message) to permit the BMS 200 to develop a behavioral signature in accordance with the present invention. Further, data captured in accordance with the present invention may be further combined with other data gathered using body-contacting devices (such as “fitness trackers” and other wearable devices 40) and/or data gathered as the results of active, purpose-specific (e.g., participation in interviews, questionnaires, etc.) and/or active, non-purpose-specific (e.g., activities of daily living, including monitoring the user's other activities, such as interacting with a computer or smartphone, e.g., to send an e-mail or text, browse the internet/Web, etc.), and the data may be provided to the BMS 200 to develop a behavioral signature in accordance with the present invention.

In accordance with the present invention, the BMS 200 further includes software employing artificial intelligence, machine learning, and/or other techniques to analyze captured bodily movements, audible vocalizations and/or other behavioral functions to identify patterns, assess characteristics, etc. that define a then-current behavioral signature for a particular person. In this manner, personal behavioral signatures, e.g., including a three-dimensional motion signature component of the person's actually observed three-dimensional bodily movements, is created for each person.

The system is then used to monitor the person to gather additional behavioral data over time. Accordingly, the present invention further provides a system and method for monitoring the person's behavioral function(s) across multiple dimensions over time, and to compare subsequently-observed behavior/behavioral signature to the person's previous behavioral (baseline) signature. The system and method compare the subsequently observed behavioral signature to the previously-obtained baseline/signature to identify a change, or delta, in the behavior, and outputs or uses the observed changes to define and evaluate disease state, measure cognitive function, detect changes in cognitive function, and/or infer attribution to changes in behavioral activity. Notably, all of this can be done passively, without disrupting the user's day-to-day activities or their use of electronic devices.

For example, a change in a person's current behavior, as observed by comparison of a later behavioral signature to a prior/baseline behavioral signature, is indicative of that person's current status as compared to a previous status. For example, the changes may be indicative of an impact of medications or other therapies for the person. Accordingly, actually-observed three-dimensional bodily movements may be used as an indicator of the person's response to treatments/therapies/medications. Accordingly, the present invention provides for objective comparison of changes in the person over time, rather than relying on subjective assessments (such as the person's responses to a healthcare provider's “How do you feel?/Do you feel better?/Do you think you've improved?/Are you still hearing voices?” inquiries, as is often the case for assessment of behavioral issues in the current state of the art). Thus, the system and method provide an objective assessment of how/whether a patient is responding to treatment, and to determine when the patient has improved sufficiently (e.g., a returned to a healthy baseline behavior, or demonstrated a significant change relative to baseline behavior), and/or to determine whether a patient's behavior has deteriorated (e.g., returned to a prior unhealthy behavior).

Further, the present invention enables the identification/creation and detection of new types of signatures of disease and risk (for instance fall risk or medication response) and also allows a person to be monitored for changes in their disease in an unobtrusive manner, to view those changes over time, and to evaluate the impact of these states. Further still, the present invention enables the identification/creation and detection of new types of drug/treatment signatures, as reflected in the bodily movements. The system and method of the present invention may develop these signatures of disease/risk/drug/treatment by monitoring an associated person's behavior over time (e.g., while using a drug or developing a disease), and/or by aggregating multiple different persons' monitored behavior over time (e.g., while using a drug or developing a disease), and using software, artificial intelligence, machine learning, and/or other techniques to analyze captured bodily movements and/or other behavioral data/functions to identify patterns, assess characteristics, etc. and to corresponding create such signatures.

One embodiment of the present invention is a method for unobtrusively recording an individual's complete activity and bodily movement within a three-dimensional environment, such as an inpatient hospital unit. In this embodiment, imaging-based units (such as Lidar-based units) are installed in selected locations and are able to track patient movements, build three dimensional maps of all activity including activity related to the movements and locations of other people, such as patients and staff members. The system can also record and analyze a voice and facial features and be connected to medical record and treatment and outcome information. The method of the present invention may include the step of recording data from devices a person wears to add additional data points such as vital signs and sleep information. While other systems have inferred data on movement and location from GPS or similar data, the system of the present invention collects actual and precise bodily movement, location and behavioral data in space, e.g., in three-dimensional space. The information and data collected can be precisely matched to various treatment and clinical data and create unique and next generation signatures of disease state.

The method of the present invention can further include the step of gathering data from an Electronic Medical Records System 120, and analysis of the electronic medical record data, e.g., the clinical notes of the staff about the patient and learning. This link allows for the identification of objective, real-time, all of the time analysis of how a patient is doing and creating predictions, based on these records, about when a patient may be ready for a treatment change or discharge even before the clinical staff sees the patient on a given day. It can also include the staff recording (e.g., at a Caregiver Computing Device 90a, 90b) what food a patient consumes as well as their weight and tying behavioral and treatment signatures to food consumption behavior and how that relates to the other bodily movement, voice and facial behaviors captured by the imaging system 50 and/or audio capture system 60. It can also be linked to other medical record data such as blood tests, vital signs, etc., e.g., as the data is provided to the BMS 200 via the Caregiver Computing Device 90a, 90b and/or the Electronic Medical Records System 120. The method of the present invention may also include the step of recording medications taken, dosages, and quantities. This information can also be correlated to changes in behavioral disease function. The method of the present invention can further include the step of recording data from wearable devices 40, e.g., at Wearables Data System 140, that measure relevant observable body or bodily function characteristics such as, by way of example, heart-rate, blood oximetry, body temperature, electroencephalogram, etc., and providing all of this data to the BMS 200 for use in developing behavioral signatures.

The data captured from the system is preferably persisted in the system's storage (e.g., at the BMS 200 or at local hardware, e.g., at the hospital 20) and then further transmitted to a cloud computing system (e.g., BMS 200) to build databases linked to data from sources such as the EMR 120 including long term outcomes and readmissions to better predict, among many other things, who should be discharged in the future, and when.

It should be noted that the imaging and audio capture systems may be provided within multiple buildings or other settings (e.g., at home and/or at a workplace, or in other public places), and that the data may be similarly aggregated for a single patient, for developing that patient's behavioral signature(s) and/or assessing how that individual's behavior changes over time. The identification and/or assessment of a change/delta in the individual's behavior is advantageous, as behavioral and medical health has historically been based on snapshots of single moments, such as “current mood” but in fact, it is the changes over time that are key for many conditions. Additionally, the system allows for identifying when the delta is relevant. Further, data may be gathered for multiple patients at a single facility (e.g., hospital), or at multiple buildings or other settings, and the data may be aggregated across multiple patients, e.g., by the BMS 200, to develop disease behavioral signatures, treatment/drug behavioral signatures, etc., by finding patterns in the behavioral signatures of multiple patients and correlating them with diseases, treatments, drugs, etc. Subsequently, for example, the BMS may be able to identify a likely disease or new disease subtypes in an observed person because of a correlation between the person's behaving and the disease's behavioral signature. By way of further example, the BMS 200 may identify whether a particular patient is improving, or developing a side-effect, as the result of a treatment/drug, because of a correlation between the person's behavior and the treatment/drug's behavioral signature.

One implementation of the present invention comprises a system and method that enables, inter alia, a passive assessment of a person's behavioral functions from contactless capture of bodily movements and sub-movements in three-dimensional space (e.g., using an imaging system such as a 3-D LIDAR or a 2-D video camera system) and/or contactless capture of vocalizations (e.g., using a microphone).

Optionally, such passively captured behavioral data may be combined with other behavioral function data that may be captured by recording on an electronic device the occurrence and timing of user events comprising the opening and closing of applications resident on the device, the characters inputted and the touch-screen gestures, tapping, body motions, and eye movements used on those applications, further recording the kinetic activities of motion, gait and balance from wearable gyroscopic and accelerometer sensors.

The data captured in the by devices 40, 50a, 50b, 60 may be transmitted to cloud computers. Transmission preferably uses a secure channel and can use hypertext transfer protocol secure (HTTPS) or another protocol to securely transfer the data. The cloud computers may analyze the recorded data against historical recordings and against recordings of other users including users that are demographically matched to the existing user. The outputs of the analyses are preferably one or more behavioral function measures. The user or the user's delegate may log into a password protected online account to view these outputs. In another embodiment of the invention the outputs of the analyses are transmitted back to the Caregiver Computing Devices.

Accordingly, the present invention comprises a system and method that enables passive and contactless monitoring of a person's bodily movements and/or vocalizations, and thus behavioral functions. Further, the present invention comprises a system and method that further performs the step of analyzing the captured data (including image data and/or audio capture data), and identifying patterns or otherwise developing a current behavioral signature for a specific person.

### Behavior Monitoring System

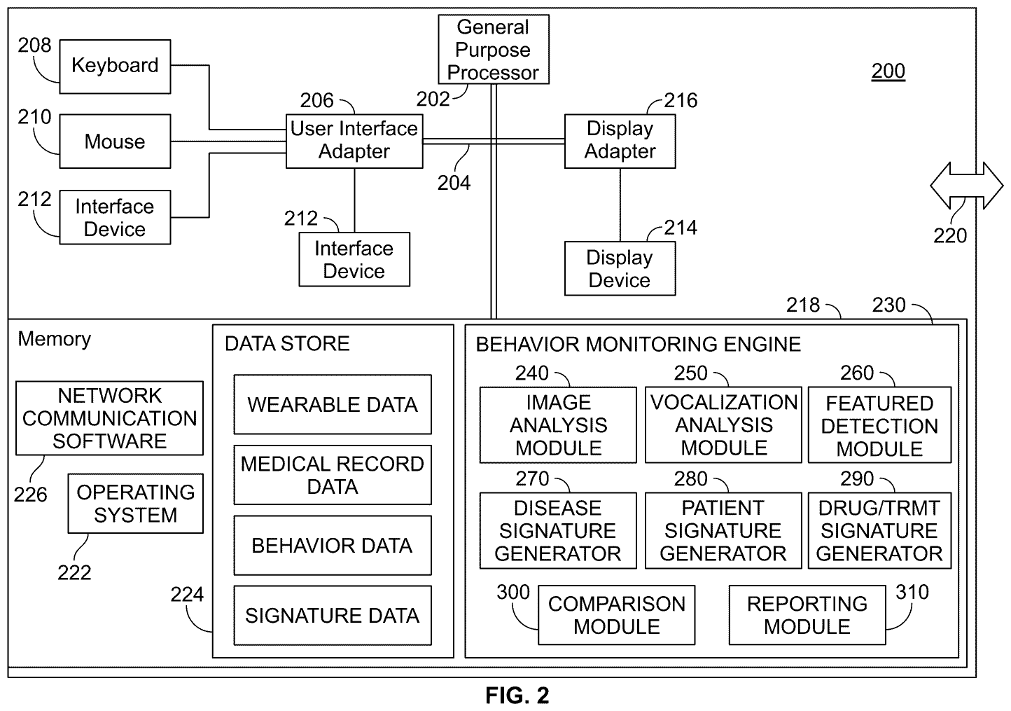

FIG. 2 is a block diagram showing an exemplary Behavior Monitoring System (BMS) 200 in accordance with an exemplary embodiment of the present invention. The BMS System 200 is a special-purpose computer system that includes conventional computing hardware storing and executing both conventional software enabling operation of a general purpose computing system, such as operating system software 222, network communications software 226, and specially-configured computer software for configuring the general purpose hardware as a special-purpose computer system for carrying out at least one method in accordance with the present invention. By way of example, the communications software 226 may include conventional web server software, and the operating system software 222 may include IOS, Android, Windows, Linux software.

Accordingly, the exemplary BMS System 200 of FIG. 2 includes a general-purpose processor, such as a microprocessor (CPU), 102 and a bus 204 employed to connect and enable communication between the processor 202 and the components of the presentation system in accordance with known techniques. The exemplary presentation system 200 includes a user interface adapter 206, which connects the processor 202 via the bus 204 to one or more interface devices, such as a keyboard 208, mouse 210, and/or other interface devices 212, which can be any user interface device, such as a touch sensitive screen, digitized entry pad, etc. The bus 204 also connects a display device 214, such as an LCD screen or monitor, to the processor 202 via a display adapter 216. The bus 204 also connects the processor 202 to memory 218, which can include a hard drive, diskette drive, tape drive, etc.

The BMS System 200 may communicate with other computers or networks of computers, for example via a communications channel, network card or modem 220. The BMS system 200 may be associated with such other computers in a local area network (LAN) or a wide area network (WAN), and may operate as a server in a client/server arrangement with another computer, etc. Such configurations, as well as the appropriate communications hardware and software, are known in the art.

The BMS System 200 is specially-configured in accordance with the present invention. Accordingly, as shown in FIG. 2, the BMS System 200 includes computer-readable, processor-executable instructions stored in the memory 218 for carrying out the methods described herein. Further, the memory 218 stores certain data, e.g. in one or more databases or other data stores 224 shown logically in FIG. 2 for illustrative purposes, without regard to any particular embodiment in one or more hardware or software components.

Further, as will be noted from FIG. 2, the BMS System 200 includes, in accordance with the present invention, a Behavior Monitoring Engine (BME) 230, shown schematically as stored in the memory 218, which includes a number of additional modules providing functionality in accordance with the present invention, as discussed in greater detail below. These modules may be implemented primarily by specially-configured software including microprocessor-executable instructions stored in the memory 218 of the BMS System 200. Optionally, other software may be stored in the memory 218 and and/or other data may be stored in the data store 224 or memory 218.

### Patient Behavioral Signature

For example, the BME 230 includes an Image Analysis Module (IAM) 240. Three-dimensional image data captured by Lidar/other imaging unit 50a (FIG. 1) and/or two-dimensional image data captured by video camera 50b (FIG. 1) may be received and stored as Behavior Data in the data store 224 of the BMS 200, as shown in FIGS. 1 and 2. Such image data may then be retrieved and processed by the IAM 240. For example, 3-D bodily movement image data may be processed by the IAM 240 to identify different angles between body parts as the person moves, different speeds of the body and its parts, etc. The way motion occurs, the speed and angles, and the changes in these can be plotted in multiple dimensions and tracked objectively consistent with the present invention. This stands in sharp contrast to traditional, subjective approaches, in which a clinician would typically ask a patient how they were feeling, and the patient might report that he/she felt low energy and felt sluggish. The present system can objectively assess patient behavior compared to other individuals as well as to the same individual's previous behavior, as identified by the person's behavioral signature(s).

Additionally, for example, a new medication may be given to the patient. The medication may change the patient's sense of balance. The signature can be used to identify changes in posture, in the angles of limb movements and other variables that can be associated with fall risk as a side effect, using objectively observable behavior of the patient, consistent with the present invention. Alternatively, 2-D bodily movement image data may be processed by the IAM 240 to have less robust but often useful 2-D signatures, such as speed of motion, but lacking in some of the angles of motion and their changes. It can also identify repeated movement patterns and new non-repeated patterns. For example, the relative amounts of repeated and chaotic patterns can be indicative of different thought states or of clinical phenomena such as akathisia. Such bodily movement image data may subsequently be used to develop or compare behavioral signatures, as described in greater detail below.

By way of further example, the BME 230 includes a Vocalization Analysis Module 250. Patient vocalizations captured by microphone 60 may be received and stored as Behavior Data in the data store 224 of the BMS 200, as shown in FIGS. 1 and 2. Such vocalization signal data may then be retrieved and processed by the Vocalization Analysis Module 240. For example, voice recognition software, speech analysis software and/or textual analysis software to analyze voice, tone, word choice, speed of speech, quantity of words, length of sentences, use of neologisms, etc. from audio signals captured by the audio capture system to provide additional personal behavioral data that may be aggregated with the personal bodily movement data to develop behavioral signatures, as described herein. This data can be associated with behavioral changes, both the content as well as the speed of speech, the paucity of spontaneous speech, the tone of speech, and the words chosen. Such vocalization data may subsequently be used to develop or compare behavioral signatures, as described in greater detail below.

By way of further example, the BME 230 also includes a Feature Detection Module 260, which uses software, artificial intelligence, machine learning, and/or other techniques to analyze captured bodily movements and/or vocalizations to identify patterns, assess characteristics, etc. that may be an indicator of certain behavioral problems or improvements. For example, with respect to bodily movements, patterns in hand movements, forearm movements, head movements may be identified. By way of further example with respect to vocalizations, patterns in voice commands, words, and phrases used may be extracted, and signal processing of the voice, recurring combinations of phones and phoneme may be extracted. Features of each pattern including voice pitch, amplitude, and frequency spectrum may be extracted from the data. In one example, the voice may capture a sound associated with surprise, and the bodily motion may indicate bumping motion indicating coordination issues caused by a new medication. In another example, the person may speak less and may have slower motion, which might indicate a depressed mood or a medication side effect.

The BME 230 also includes a Patient Signature Generator 280. The Patient Signature Generator analyzes patterns detected by the Feature Detection Module 260, and then defines a then-current behavioral signature for a particular person. In this manner, personal behavioral signatures, e.g., including a three-dimensional motion signature component of the person's actually observed three-dimensional bodily movements, is created for each person. For example, a medication may cause tardive dyskinesia as a side effect, and this could be seen in the system as a change in the patient's objectively observable behavior. It may also capture facial changes, such as blunted affect. In addition, it may show increased physical activity associated with a medication, such as an anti-depressant. The system may also find that certain repeated whole body or partial body movements are associated with certain medical states or changes in those states. For instance, giving a sedative may have a certain set of changes such as more gross movements but fewer fine muscle movements. The particular patient's behavioral signature may then be stored as behavior data in the Data Store 224 for future reference.

Multiple then-current patient behavior signatures may be generated over time. The BME 230 further includes a Comparison Module 300. In accordance with one aspect of the present invention, the Comparison Module 300 may be used to compare a patient's current behavioral signature with a prior behavioral signature. For example, the Comparison Module 300 may indicate that the behavioral signatures are the same or similar. The comparison may be performed by software, artificial intelligence, machine learning, and/or other techniques to analyze a relationship between the signatures. In a certain context, the same or similar behavioral signatures may indicate a lack of improvement, or a lack of deterioration in behavior. In a certain context, the same or a similar behavioral signature may indicate a recurrence of a prior behavioral problem, or a cease of taking of certain medication for treating the behavioral problem, or a failure of a certain medication to effectively treat the behavioral problem. The Comparison Module 300 may take into account Medical Record Data received from Caregiver Computing Devices 90a, 90b and/or the patient's medical records as retrieved from an Electronic Medical Records System 120 and stored as Medical Record Data in the Data Store 224. By way of example, artificial intelligence and other analyses comparing behavioral data captured from the system can be examined in the context of past and present and future medical record data to identify signals or clinical change or risk or improvement, such as fewer hospitalizations, improved functioning as described by patients and clinicians, and lower cost of care. It may also be associated with certain medication changes.

Further, the Comparison Module 300 may take into account “wearable device” data received from a wearables device 40 and/or as retrieved from a Wearables Data System 140 and stored as Wearable Data in the Data Store 224. Recordings from wearable electronic devices that measure heart rate, blood pressure, pulse oximetry, body temperature, and other physiological data may be recorded. The peripheral accessories can be used to obtain biological vital signs of the individual, which can be used to determine if a decline in behavioral function data is due to a vital sign such as fatigue or low blood-glucose levels rather than an actual decline in behavioral function of the individual. The biological vitals can also be used as an alert of a biological trend that will have a long-term negative impact on behavioral function. For example, patterns in heart rate, blood pressure, body temperature, blood oximetry and other physiologic measurements may be extracted from the data. Features of each pattern including maximum and minimum measurements, duration of pattern, frequency of pattern, are also preferably extracted.

Results/conclusions or notices of the Comparison Module 300 may be output by the Reporting Module 310. For example, the Reporting Module may provide a communication or alert to a Caregiver Computing Device 90a, 90b, or send an electronic message or other communication to a healthcare provider, or record notes in the EMR System 120, or otherwise be used to support the making of a diagnosis or assessment of the patient and/or the patient's behavioral health.

It should be noted that the behavioral signature data and comparisons described herein are useful in supporting diagnosis or assessment of the patient and/or the patient's behavioral health, by providing objectively observable insights and analyses. The data so gathered may also advantageously be combined with other data to support the making of a diagnosis or assessment of the patient and/or the patient's behavioral health. Such other data may come from various sources, including data from the EMR System 120 and/or data from a wearable device of system, as data from these systems may inform such diagnoses and assessments. By way of example, wearable devices may capture data relating to blood pressure, body temperature, sweat, sweat content, sleep, and the like.

### Disease Behavioral Signature

As described above, personal behavior can be monitored in a passive, contactless fashion, and personal behavioral signatures can be developed and compared. In accordance with another aspect of the present invention, the BME 230 of the BMS 200 includes a Disease Signature Generator 270, and is capable of developing behavioral signatures of a disease. More particularly, in accordance with this aspect of the present invention, the Feature Detection Module is operable to detect behavioral patterns and/or characteristics (collectively, “patterns”) across behavior or behavioral signatures of a plurality of individuals. The plurality of individuals may be defined as a population diagnosed as having a common disease, e.g., as may be reflected in Medical Record Data stored in the Data Store 224 or reflected in records of an EMR System 120. By analyzing behavior (raw data or behavioral signatures) of multiple individuals sharing a common disease, the Feature Detection Module can identify patterns, and the Disease Signature Generator 270 can develop a signature representing common characteristics representative of the disease, as evidenced in commonality or other patterns in the observed behavior data. Such disease signature data may be stored as Signature Data in the Data Store 224.

In accordance with this aspect of the present invention, the Comparison Module 300 may subsequently be used to compare an individual's personal behavioral signature to disease signatures stored in the Data Store 224, and the Reporting Module 310 may be used to provide output indicated whether there is sufficient correlation between the patient's behavior and a disease's characteristic behavior, as reflected in the disease signature data, such that the patient may be considered to have the disease, or be evaluated for having the disease. Accordingly, patterns of disease behavior in populations may be detected, and may be used to assess or predict disease state in a particular individual in a manner that is highly sensitive, specific, and unobtrusive to an individual.

### Drug/Treatment Behavioral Signature

As described above, personal behavior is monitored in a passive, contactless fashion, and personal behavioral signatures can be developed and compared. In accordance with another aspect of the present invention, the BME 230 of the BMS 200 includes a Drug/Treatment Signature Generator 290, and is capable of developing behavioral signatures of a drug or treatment as well as identifying changes in the behavior when treatment is changed, initiated or ended. For example, certain signatures may be used to identify effect of a drug, side effects of a drug, or ineffectiveness of a treatment. More particularly, in accordance with this aspect of the present invention, the Feature Detection Module is operable to detect behavioral patterns across behavior or behavioral signatures of a plurality of individuals. The plurality of individuals may be defined as a population received a particular drug or treatment, e.g., as may be reflected in Medical Record Data stored in the Data Store 224 or reflected in records of an EMR System 120. By analyzing behavior of multiple individuals sharing a common treatment, the Feature Detection Module can identify patterns, and the Disease Signature Generator 270 can develop a signature representing common characteristics representative of the drug/treatment, as evidenced in commonality or other patterns in the observed behavior data. Such drug/treatment signature data may be stored as Signature Data in the Data Store 224.

In accordance with this aspect of the present invention, the Comparison Module 300 may subsequently be used to compare an individual's personal behavioral signature to disease and/or drug/treatment signatures stored in the Data Store 224, and the Reporting Module 310 may be used to provide output indicated whether there is sufficient correlation between the patient's behavior and a disease and/or drug/treatment's associated characteristic behavior, as reflected in the drug/treatment signature data, such that the patient may be considered to well-suited to receiving the drug/treatment, or be improving with the drug/treatment, or be deteriorating while receiving the drug/treatment. Accordingly, patterns of drug/treatment behavior in populations may be detected, and may be used to assess or predict drug/treatment efficacy in a particular individual in a manner that is highly sensitive, specific, and unobtrusive to an individual.

### System Functionality

In one implementation of the method and system, in order to establish a baseline of data, supervised benchmark behavioral monitoring can be conducted on an initial test group of individuals, and the data is stored. Data for each individual can be recorded as outlined herein, and the data can be correlated to benchmark testing results and behavioral function. Behavioral function levels and bands may also be determined from the result. Once certain baselines have been established and correlations are made, behavioral function may be utilized to improve the system and method as learning occurs. The learning from the subsequent mobile device usage may be considered unsupervised learning.

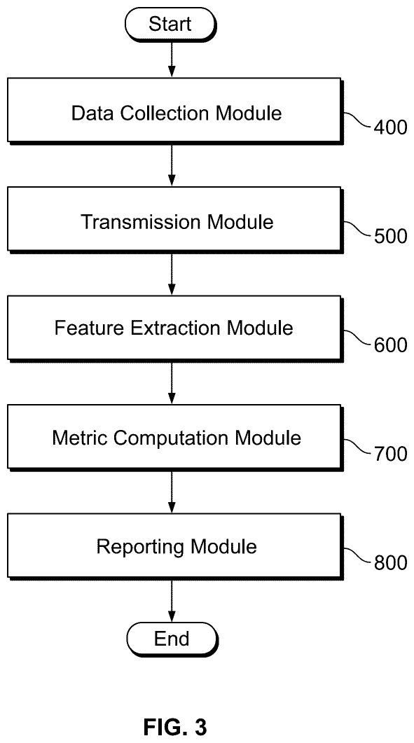

FIG. 3 illustrates an embodiment of the functional description of the system configured in accordance with the present invention and is not intended to limit scope, as one of ordinary skill would understand on review of this application that other configurations could be utilized without departing from the scope of the claimed invention. Referring to FIG. 3, a user's bodily movements and/or vocalizations may be captured and recorded by data collection modules 400 resident on the device imaging 50a, 50b and/or microphone 60 devices. A transmission module 500 may be resident on the device and responsible for transmitting and receiving data to and from local computers, hardware and/or cloud computers. This module preferably uses broadband WIFI for the transmission when available, but may alternatively use other transmission means including 4G LTE transmission provided by subscriber data plans. Transmission module 500 preferably may be responsible for securing an encrypted channel.

The feature extraction module 600 shown in FIG. 3 is part of the Feature Detection Module 260 of FIG. 2, and may extract patterns and features from the data acquired by one or more of the imaging and audio capture devices. In one embodiment of the present invention, feature module extraction 600 is resident on cloud computers, such as BMS 200. In another embodiment of the present invention, feature extraction module 600 is resident on the device itself, and features are extracted from data acquired by data collection module 400 on that device. The metric computation module 700 may develops behavioral function measures that it takes as input features from feature extraction module 600. Metric computation module 700 is part of the Patient Signature Generator 280. In one embodiment of the present invention, the predictive models of metric module computation 700 are resident on cloud computers, such as BMS 200. The metric computation model 700 may also be responsible for learning the predictive models of behavioral function measures from a population of users for which both feature data and traditional behavioral function testing data is available.

A reporting module 800 preferably provides an online login account for the user or the user's delegate to review trends in the user's behavioral function measures, how well the user is tracking to target behavior activity, and further may enable the user to update those targets. Metric computation module 700 is part of the Reporting Module 310.

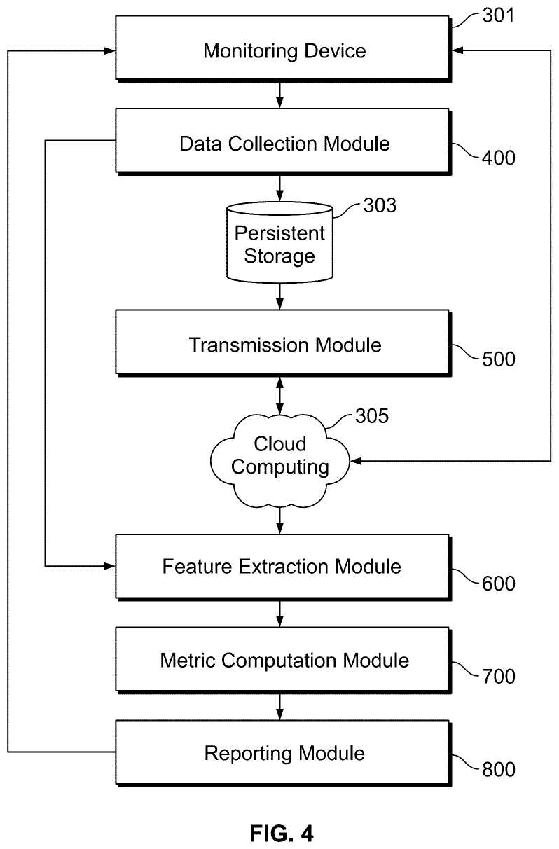

FIG. 4 illustrates an embodiment of the computing environment of the system configured in accordance with the present invention and is not intended to limit scope as one of ordinary skill would understand on review of this application that other configurations could be utilized without departing from the scope of the claimed invention. Referring to FIG. 4, the data collection module 400 and the transmission module 500 are integrated into or in communication with the monitoring devices 50a, 50b, 60. The data collection module 400 may write raw activity data to the persistent storage of the monitoring devices. The transmission module 500 may transmit the data to the cloud computers 305, e.g., BMS 200. The feature extraction module 600, metric computation module 700, and reporting module 800 are preferably resident in the cloud computers 305. Feature extraction module 600 may extract patterns and features from the data that are used as inputs to the metric computation module 700. The metric computation module 700 may output the behavioral function measures. The reporting module 800 may create summary reports presented to the user or the user's delegate in a password-protected account on the cloud servers.

Generally, the feature extraction module and computation module (and the associated modules as shown in FIG. 2) use machine learning, and artificial intelligence techniques to perform the image analysis, vocalization analysis, feature detection, signature generation and comparison functions described above. Any suitable techniques and approaches may be used.

By way of example, patterns, features and attributes may include extracted patterns from collected data including features such as limb movement, limb speed, limb angles, bodily movement, bodily speed, gait, duration of movement, time of movement, facial movements, location of movement, proximity to others, vocalizations, voice, word selection and other aspects described above.

By way of non-limiting example, statistics may be computed from features and attributes of patterns extracted from capture of bodily motions, vocalizations, etc., as described above. Features statistics may be computed as part of the comparison. The statistics may be computed on the raw feature values or after functional transformation of the feature values. The computed statistics may also be computed on all available values of the feature or transformed feature, or they may be computed on a subset of values filtered by one or more attributes. The statistics computed may include, for example, mean, median, mode, variance, kurtosis, moments, range, standard deviation, quantiles, inter-quantile ranges, and distribution parameters under different distributions such as exponential, normal, or log-normal. Each computed statistic for each pattern, feature, and attribute combination may be outputted. Feature statistics may be indexed by user, pattern, feature, statistic, and attribute for a population.

Machine learning methods may be used to discern a normal or characteristic behavioral signature of an individual. Various machine learning methods may be suitable as any one of ordinary skill in the art will appreciate.

By way of alternative example, the feature extraction module and computation module may use various approaches to analyze system-observed behaviors and/or behavioral patterns and to perform a health state classification matching the observed behaviors/patterns to one or more health states. For example, classification can be performed with established statistical algorithms and methods well-known in the art, useful as models or useful in designing predictive models, can include but are not limited to: analysis of variants (ANOVA); Bayesian networks; boosting and Ada-boosting; bootstrap aggregating (or bagging) algorithms; decision trees classification techniques, such as Classification and Regression Trees (CART), boosted CART, Random Forest (RF), Recursive Partitioning Trees (RPART), and others; Curds and Whey (CW); Curds and Whey-Lasso; dimension reduction methods, such as principal component analysis (PCA) and factor rotation or factor analysis; discriminant analysis, including Linear Discriminant Analysis (LDA), Eigengene Linear Discriminant Analysis (ELDA), and quadratic discriminant analysis; Discriminant Function Analysis (DFA); factor rotation or factor analysis; genetic algorithms; Hidden Markov Models; kernel based machine algorithms such as kernel density estimation, kernel partial least squares algorithms, kernel matching pursuit algorithms, kernel Fisher's discriminate analysis algorithms, and kernel principal components analysis algorithms; linear regression and generalized linear models, including or utilizing Forward Linear Stepwise Regression, Lasso (or LASSO) shrinkage and selection method, and Elastic Net regularization and selection method; glmnet (Lasso and Elastic Net-regularized generalized linear model); Logistic Regression (LogReg); meta-learner algorithms; nearest neighbor methods for classification or regression, e.g. Kth-nearest neighbor (KNN); non-linear regression or classification algorithms; neural networks; partial least square; rules based classifiers; shrunken centroids (SC): sliced inverse regression; Standard for the Exchange of Product model data, Application Interpreted Constructs (StepAIC); super principal component (SPC) regression; and, Support Vector Machines (SVM) and Recursive Support Vector Machines (RSVM), among others. Additionally, clustering algorithms as are known in the art can be useful in determining subject sub-groups.

The various implementations and examples shown above illustrate a method and system for assessing behavioral function using an electronic device. As is evident from the foregoing description, certain aspects of the present implementation are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present implementation. Accordingly, the specification and drawings are to be regarded in an illustrative rather than a restrictive sense.

Certain systems, apparatus, applications or processes are described herein as including a number of modules. A module may be a unit of distinct functionality that may be presented in software, hardware, or combinations thereof. When the functionality of a module is performed in any part through software, the module includes a computer-readable medium. The modules may be regarded as being communicatively coupled. The inventive subject matter may be represented in a variety of different implementations of which there are many possible permutations.

The methods described herein do not have to be executed in the order described, or in any particular order. Moreover, various activities described with respect to the methods identified herein can be executed in serial or parallel fashion. In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

In an exemplary embodiment, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a server computer, a client computer, a personal computer (PC), a tablet PC, a set-top box (STB), a Personal Digital Assistant (PDA), a cellular telephone, a smart phone, a web appliance, a network router, switch or bridge, or any machine capable of executing a set of instructions (sequential or otherwise) that specify actions to be taken by that machine or computing device. Further, while only a single machine is illustrated, the term “machine” shall also be taken to include any collection of machines that individually or jointly execute a set (or multiple sets) of instructions to perform any one or more of the methodologies discussed herein.

The example computer system and client computers include a processor (e.g., a central processing unit (CPU) a graphics processing unit (GPU) or both), a main memory and a static memory, which communicate with each other via a bus. The computer system may further include a video/graphical display unit (e.g., a liquid crystal display (LCD) or a cathode ray tube (CRT)). The computer system and client computing devices also include an alphanumeric input device (e.g., a keyboard or touch-screen), a cursor control device (e.g., a mouse or gestures on a touch-screen), a drive unit, a signal generation device (e.g., a speaker and microphone) and a network interface device.

The system may include a computer-readable medium on which is stored one or more sets of instructions (e.g., software) embodying any one or more of the methodologies or systems described herein. The software may also reside, completely or at least partially, within the main memory and/or within the processor during execution thereof by the computer system, the main memory and the processor also constituting computer-readable media. The software may further be transmitted or received over a network via the network interface device.

The term “computer-readable medium” should be taken to include a single medium or multiple media (e.g., a centralized or distributed database, and/or associated caches and servers) that store the one or more sets of instructions. The term “computer-readable medium” shall also be taken to include any medium that is capable of storing or encoding a set of instructions for execution by the machine and that cause the machine to perform any one or more of the methodologies of the present implementation. The term “computer-readable medium” shall accordingly be taken to include, but not be limited to, solid-state memories, and optical media, and magnetic media.

What is claimed is:

1. A computer-implemented method for capturing behavioral patterns and assessing health status via a computerized behavioral monitoring system having at least one processor and a memory operatively coupled to the memory and storing instructions executable by the processor, the method comprising:
monitoring a person's bodily behavior within an environment using a passive monitoring device;
transmitting data from said monitoring device to a computing system comprising a processor executing an analysis function for analysis of transmitted monitoring data; and
determining, by said computing system, a behavioral signature that is representative of the person's bodily behavior with the environment, by performing said analysis function, said behavioral signature representing objectively observable characteristics of bodily behavior of said person.

2. The computer-implemented method of claim 1, wherein said behavior signature represents observable characteristics of the person's bodily motions in three-dimensional space.

3. The computer-implemented method of claim 1, wherein said behavior signature represents observable characteristics of the person's audible vocalizations.

4. The computer-implemented method of claim 1, wherein said behavior signature represents observable characteristics of the person's facial muscle movements.

5. The computer-implemented method of claim 1, further comprising:
comparing, by said computing system, said behavioral signature to other signature data; and
determining, by said computing system, a behavioral state of said person as a result of said comparing.

6. The computer-implemented method of claim 5, wherein said monitoring continues over time, said method further comprising computing multiple behavioral signatures for said person over time, and wherein comparing said behavioral signature to other signature data comprises comparing a behavioral signature of said person at one point in time to another behavioral signature of said person at another point in time.

7. The computer-implemented method of claim 1, wherein said monitoring continues over time, said method further comprising computing multiple behavioral signatures for said person over time, and wherein comparing said behavioral signature to other signature data comprises comparing a behavioral signature of said person another behavioral signature of another person.

8. The computer-implemented method of claim 1, wherein the passive monitoring device comprises an imaging device.

9. The computer-implemented method of claim 8, wherein the passive monitoring device is contactless.

10. The computer-implemented method of claim 8, wherein the passive monitoring device is essentially contactless, in that it requires the person to wear one or more imaging markers.

11. The computer-implemented method of claim 8, wherein the imaging device comprises a Lidar imaging unit for capturing three-dimensional motion data.

12. The computer-implemented method of claim 8, wherein the imaging device comprises a video camera imaging unit for capturing two-dimensional motion data.

13. The computer-implemented method of claim 8, wherein said analysis function is configured to analyze at least one of: an amount of movement, a distance moved, a speed of bodily movement, facial movements, a speed of movement of a body part, a duration of movement, a frequency of movement, a sleep state, bathroom visit data, a time of day of movement, a pattern of bodily movement in the environment, a pattern of bodily motions, a repetition of movements, a repetition of motions, an interaction with other individuals in the space, and a proximity to others in the environment.

14. The computer-implemented method of claim 1, wherein said analysis function is configured to analyze information gathered from at least one of: a smartphone, a tablet computer, a wearable electronic device, and a household electronic device.

15. The computer-implemented method of claim 1, wherein said analysis function is configured to analyze information gathered from an electronic medical records system.

16. The computer implemented method of claim 15, wherein said analysis function is configured to analyze electronic medical record information comprising at least one of: a physiology measurement and a biological measurement.

17. The computer-implemented method of claim 16, wherein said analysis function is configured to analyze at least one of the following biological measurements: a Chem 7 finding, a CBC findings, a heart rate, a blood pressure, a blood oximetry, a blood glucose, a body temperature, a body fat, a body weight, a sleep duration, a sleep quality, and an electroencephalogram.

18. The computer implemented method of claim 15, wherein said analysis function is configured to analyze electronic medical record information relating to use of medications and substances with behavioral or cognitive effects selected from the group consisting of: cocaine, opiates, amphetamines, stimulants and cannabis.

19. The computer implemented method of claim 15, wherein said analysis function is configured to analyze electronic medical record information relating to food and diet information.

20. The computer implemented method of claim 15, wherein said analysis function is configured to analyze electronic medical record information relating a medication measurement selected from the group consisting of: a dosage, a frequency, and a duration of a medication.

21. The method of claim 1, wherein the passive monitoring device comprises an audio capture device.

22. The method of claim 20, wherein said analysis function is configured to analyze at least one of: words and language used in clinical interactions, words and language used in non-clinical interactions, presence of voice, tone of voice, word choice, length of words chosen, speed of speech, quantity of words, length of sentences, and use of neologisms.

23. A computer-implemented method for capturing behavioral patterns and assessing health status via a computerized behavioral monitoring system having at least one processor and a memory operatively coupled to the memory and storing instructions executable by the processor, the method comprising:
monitoring bodily behavior within at least one environment using at least one passive and contactless monitoring device for a plurality of persons;
transmitting data from said at least one monitoring device to a computing system comprising a processor executing an analysis function for analysis of transmitted monitoring data; and
computing, by said computing system for each of said plurality of persons, a respective behavioral signature that is representative of each respective person's behavior, by performing said analysis function, each said behavioral signature representing objectively observable characteristics of bodily behavior of each respective person.

24. The computer-implemented method of claim 23, wherein each respective behavior signature represents observable characteristics of each respective person's bodily motions in three-dimensional space.

25. The computer-implemented method of claim 23, wherein each respective behavior signature represents observable characteristics of each respective person's audible vocalizations.

26. The computer-implemented method of claim 23, further comprising:
comparing, by said computing system, respective behavioral signatures of said plurality of persons, said plurality of persons being a population of persons sharing a common behavioral disease; and
computing, by said computing system, a respective disease behavioral signature that is representative of commonality of the behavioral signatures of said plurality of persons, by performing said analysis function, the disease behavioral signature representing objectively observable shared characteristics of bodily behavior of said plurality of persons.

27. The computer-implemented method of claim 26, further comprising:
monitoring an other person's bodily behavior within an environment using a passive and contactless monitoring device;
transmitting data from said monitoring device to a computing system comprising a processor executing an analysis function for analysis of transmitted monitoring data;
computing, by said computing system, a behavioral signature that is representative of the other person's behavior, by performing said analysis function, said behavioral signature representing objectively observable characteristics of bodily behavior of said another person;
comparing, by said computing system, said behavioral signature of said another person to said disease behavioral signature; and
determining, by said computing system, a health state of said another person as a result of said comparing of said another person's behavioral signature to said disease behavioral signature.

28. The computer-implemented method of claim 27, wherein determining said health state of said another person comprises determining presence of absence of a disease.

29. The computer-implemented method of claim 23, further comprising:
comparing, by said computing system, respective behavioral signatures of said plurality of persons, said plurality of persons being a population of persons receiving a common medical treatment; and
computing, by said computing system, a respective treatment behavioral signature that is representative of commonality of the behavioral signatures of said plurality of persons, by performing said analysis function, the treatment behavioral signature representing objectively observable shared characteristics of bodily behavior of said plurality of persons.

30. The computer-implemented method of claim 29, further comprising:
monitoring an other person's bodily behavior within an environment using a passive and contactless monitoring device;
transmitting data from said monitoring device to a computing system comprising a processor executing an analysis function for analysis of transmitted monitoring data;
computing, by said computing system, a behavioral signature that is representative of the other person's behavior, by performing said analysis function, said behavioral signature representing objectively observable characteristics of bodily behavior of said another person;
comparing, by said computing system, said behavioral signature of said another person to said treatment behavioral signature; and
determining, by said computing system, a health state of said another person as a result of said comparing of said another person's behavioral signature to said treatment behavioral signature.

31. The computer-implemented method of claim 30, wherein determining said health state of said another person comprises determining whether a medical treatment for the person is effective or ineffective.
